(19) United States
(12) Patent Application Publication
(10) Pub. No.: US 2024/0062891 A1
(43) Pub. Date: Feb. 22, 2024

(54) ACTION ORIENTED MEDICAL GUIDANCE SYSTEM

(71) Applicant: QuantGene Inc., Santa Monica, CA (US)

(72) Inventor: Johannes Suriya Bhakdi, Vancouver, WA (US)

(21) Appl. No.: 18/236,254

(22) Filed: Aug. 21, 2023

Related U.S. Application Data
(60) Provisional application No. 63/399,700, filed on Aug. 21, 2022.

(51) Int. Cl. G16H 40/63; G16H 10/60; G16H 40/20; G16H 40/40; G16H 50/30; A61B 5/00; A61B 5/0205; G16H 20/00; A61B 5/021; G16H 50/20

(52) U.S. Cl. CPC G16H 40/63; G16H 10/60; G16H 40/20; G16H 40/40; G16H 50/30; A61B 5/681; A61B 5/0205; G16H 20/00; A61B 5/7435; A61B 5/02141; G16H 50/20

(57) ABSTRACT

Methods, systems and apparatus for providing action based medical guidance. The system may receive user data through a user interface or action item user interface. The guidance system may receive patient data including comprehensive personal history, a comprehensive family history, third party medical records, total genomics records, non-genomics diagnostic and testing results and generate medical guidance based on the analysis of the received patient data. User's may be notified of the guidance through the user interface.

### CROSS-REFERENCE TO RELATED APPLICATIONS

This application claims the benefit of priority to U.S. Provisional Application No. 63/399,700, filed Aug. 21, 2022, which is hereby incorporated by reference in its entirety.

### FIELD

The present invention relates generally to the analysis of screening or test results and the generation of recommendations and insight based off of the analysis.

### BACKGROUND

Modern day patients wish to be more informed when it comes to their health. Current systems do not provide patients with the amount of information or the professional insight that could aid in helping a patient to understand their own body, condition and treatment.

### SUMMARY

The systems and methods described herein provide for a computer implemented medical action guidance system and action item user interface. The guidance system may be configured for receiving, for a patient, patient data from a data input module. In some embodiments, the patient data may include a comprehensive personal history, a comprehensive family history, third party medical records, total genomics records, non-genomics diagnostic records or combination thereof. In some embodiments, a medical intelligence module may be configured to analyze the received patient data. Analyzing may be performed by a machine learning model. The machine learning model may be trained, by a training module, on labeled datasets comprising historic patient data, medical recommendations and physician decisions.

In some embodiments, the medical intelligence module may generate one or more medical recommendations based on the analysis of the patient data. The one or more medical recommendations and the patient data used in the generating of the one or more medical recommendations may then be provided to a medical group or medical group module. The system may receive, from the medical group module, a physician decision for each of the one or more medical recommendations.

In some embodiments, the received patients data, the one or more medical recommendations and the physician decisions may be stored in a datastore. The stored information may then be used in the training and retraining of the machine learning model.

In some embodiments, the analyzed patient data may be sent to a user interface. The by the user interface may then display one or more action items associated with the patient. The one or more action items may comprise a visual representation of priority, status, and the analyzed patient data. In some embodiments, the action items may be ordered in the user interface based on priority and status. The user interface may further be configured to schedule an appointment for the patient. The appointment corresponding to a selected action item selected by the patient.

In some embodiments, the comprehensive personal history and the comprehensive family history may be obtained from the patient through a user interface based patient intake form.

In some embodiments, the medical intelligence module may be configured to classify, by a risk profile module, a patient risk profile. The risk profile corresponding to one of a plurality of risk categories. The one or more medical recommendations provided to the medical group module may include the classified patient risk profile.

In some embodiments, the visual representation of status corresponds to a completion state of a procedure and a number of days until a due date of the action item.

In some embodiments, the action item user interface may be configured configured to receiving, for a patient, analyzed patient data from a medical intelligence module operating on a server.

One or more medical condition or disorder diagnoses may be determined or received for the patient based at least in part on the analyzed data. One or more medical interventions for each medical condition or disorder may be determined or received, wherein the medical interventions comprise procedures, tests, screenings and/or courses of action.

In some embodiments, a risk profile for the patient may be determined. The risk profile may comprise a risk category for each of the patient's medical conditions or disorders. A recommended due date for each of the procedures, tests, screenings and courses of action associated with the patient's medical condition or disorder may then be determined. The recommended due date determination may be based on the analyzed patient data.

In some embodiments, the user interface may be configured to create one or more action items to be displayed. Each action item may be associated with a medical condition or disorder, a medical intervention, a risk category and/or a recommended due date. The user interface may then display the one or more action items associated with the patient. The action items comprise a visual representation of priority, status, risk category, recommended due date and/or an indication of the an amount of time until the recommended due date and/or an indication of how overdue a patient action is based on the recommended due date.

In some embodiments, the user interface may be configured to receive, from a patient, a selection of a displayed action item. A detailed view of the selected action item may then be display to the patient. In some embodiments, a patient may initiate a scheduling process associated with the corresponding action item's medical condition or disorder through the detailed view of the selected action item.

The features and components of these embodiments will be described in further detail in the description which follows. Additional features and advantages will also be set forth in the description which follows, and in part will be implicit from the description, or may be learned by the practice of the embodiments. The detailed description and specific examples are intended for illustration only and are not intended to limit the scope of the disclosure

### BRIEF DESCRIPTION OF THE DRAWINGS

The present disclosure will become better understood from the detailed description and the drawings, wherein:

FIG. 1 is a diagram illustrating an exemplary action oriented medical guidance platform in which some embodiments may operate.

FIG. 2A is a diagram illustrating an exemplary patient interface in accordance with aspects of the present disclosure.

FIG. 2B is a diagram illustrating an exemplary data input system in accordance with aspects of the present disclosure.

FIG. 2C is a diagram illustrating an exemplary medical intelligence server in accordance with aspects of the present disclosure.

FIG. 2D is a diagram illustrating an exemplary services server in accordance with aspects of the present disclosure.

FIG. 3A is a diagram illustrating an exemplary user interface in accordance with some embodiments.

FIG. 3B is a diagram illustrating an exemplary user interface in accordance with some embodiments.

FIG. 3C is a diagram illustrating an exemplary user interface in accordance with some embodiments.

FIG. 3D is a diagram illustrating an exemplary user interface in accordance with some embodiments.

FIG. 3E is a diagram illustrating an exemplary user interface in accordance with some embodiments.

FIG. 3F is a diagram illustrating an exemplary user interface in accordance with some embodiments.

FIG. 3G is a diagram illustrating an exemplary user interface in accordance with some embodiments.

FIG. 3H is a diagram illustrating an exemplary user interface in accordance with some embodiments.

FIG. 3I is a diagram illustrating an exemplary user interface in accordance with some embodiments.

FIG. 3J is a diagram illustrating an exemplary user interface in accordance with some embodiments.

FIG. 3K is a diagram illustrating an exemplary user interface in accordance with some embodiments.

FIG. 3L is a diagram illustrating an exemplary user interface in accordance with some embodiments.

FIG. 3M is a diagram illustrating an exemplary user interface in accordance with some embodiments.

FIG. 3N is a diagram illustrating an exemplary user interface in accordance with some embodiments.

FIG. 3O is a diagram illustrating an exemplary user interface in accordance with some embodiments.

FIG. 4A is a flow chart illustrating an exemplary method that may be performed in accordance with some embodiments.

FIG. 4B is a flow chart illustrating an exemplary method that may be performed in accordance with some embodiments.

FIG. 5 is a diagram illustrating an exemplary computer/control system that may perform processing in some embodiments and in accordance with aspects of the present disclosure.

### DETAILED DESCRIPTION

In this specification, reference is made in detail to specific embodiments of the invention. Some of the embodiments or their aspects are illustrated in the drawings.

For clarity in explanation, the invention has been described with reference to specific embodiments, however it should be understood that the invention is not limited to the described embodiments. On the contrary, the invention covers alternatives, modifications, and equivalents as may be included within its scope as defined by any patent claims. The following embodiments of the invention are set forth without any loss of generality to, and without imposing limitations on, the claimed invention. In the following description, specific details are set forth in order to provide a thorough understanding of the present invention. The present invention may be practiced without some or all of these specific details. In addition, well known features may not have been described in detail to avoid unnecessarily obscuring the invention.

In addition, it should be understood that steps of the exemplary methods set forth in this exemplary patent can be performed in different orders than the order presented in this specification. Furthermore, some steps of the exemplary methods may be performed in parallel rather than being performed sequentially. Also, the steps of the exemplary methods may be performed in a network environment in which some steps are performed by different computers in the networked environment.

Some embodiments are implemented by a computer system. A computer system may include a processor, a memory, and a non-transitory computer-readable medium. The memory and non-transitory medium may store instructions for performing methods and steps described herein.

The following generally relates to a system, platform, user interface and methods for an action oriented medical guidance system. In some embodiments, the system may receive patient data from one or more sources and then analyze the received data with one or more machine learning models. The analysis by the one or more machine learning models may include generating one or more risk profiles for a patient and/or generating one or more recommendations relating to the patient.

In some embodiments, the patient data may include comprehensive medical history of both the patient and their family. In some embodiments, the patient may provide a sample for genetic testing. The sample may be sequenced, and said sequence analyzed to identify one or more variants. The system may then be configured to determine pathogenicity for the identified variants in the sequence. The analysis may be performed by the one or more machine learning models. In some embodiments, a model may be used to identify variants in the genetic sequence and a separate model may be used to determine the pathogenicity of the identified variants. In some embodiments, the identification of variants and determination of pathogenicity may be carried out by the same machine learning model. The system may then generate one or more risk profiles based on the determined pathogenicity.

The generated risk profiles may then be transferred to an operations server, where a medical group may review the generated risk profiles. One or more physicians or medical practitioners may then approve or reject the risk profiles based on review of the risk profiles the data used in generating the risk profile. Approved risk profiles may then be sent to a graphical user interface to be displayed to the patient.

In some embodiments, the user interface may be accessed by a patient through a mobile application operating on a patient's smartphone, tablet or other electronic device. The user interface may also be in the form of a web application, accessed and used through a web browser. In some embodiments, the risk profiles received by the user interface may be used to create action items and care recommendations. In some embodiments, each risk profile may correspond to a single action item. Visual representations of the action items may be displayed to the patient through the user interface. Each action item may have a care recommendation associated with it. The patient may select and review the any action item displayed. While reviewing the action item, the patient may be able to schedule an appointment corresponding to the care recommendation of the action item.

In some embodiments, when a procedure related to the action item care recommendation is completed by the patient, the status of the action item may be changed. Changes to the status of an action item may also result in a change to a visual representation of the action item in the user interface. In some embodiments, the patient may upload results from the procedure or the results may be transferred automatically to the system. The user interface may be configured to show the status of the action item as being “in review” after the results have been uploaded/transferred. The status may remain as “in review” until such time as a final outcome is determined. In some embodiments, the uploaded/transferred results may be analyzed by a machine learning model to determine an outcome of the action item. A physician may then review the determined outcome and the uploaded/transferred results, and confirm or reject the determination. The status of the action item may be updated based on the determined outcome. A visual representation of the outcome may be displayed for the action item, wherein the outcome representation is color coded according to the determined outcome. In some embodiments, each status and outcome may be associated with a color. For example, a status of “in review” may be visually represented by the color blue, a positive result to a test may be represented as red and negative test results may be represented by the color green.

In some embodiments, the machine learning model is configured to continually learn every time a physician makes a decision or confirms/rejects a decision of the machine learning model.

In some embodiments, the user interface may provide a graphical representation of each action items. The graphical representation may be configured to visually convey importance, status, and/or priority of the action item in an intuitive manner. For example, important action items, or action items that are overdue may be colored red to draw a patient's attention. In some embodiments, a countdown may be displayed for each action item. The countdown may show the number of days until an action item needs to be completed or until an appointment date. The countdown may be text, progress bar or other visual representations of time.

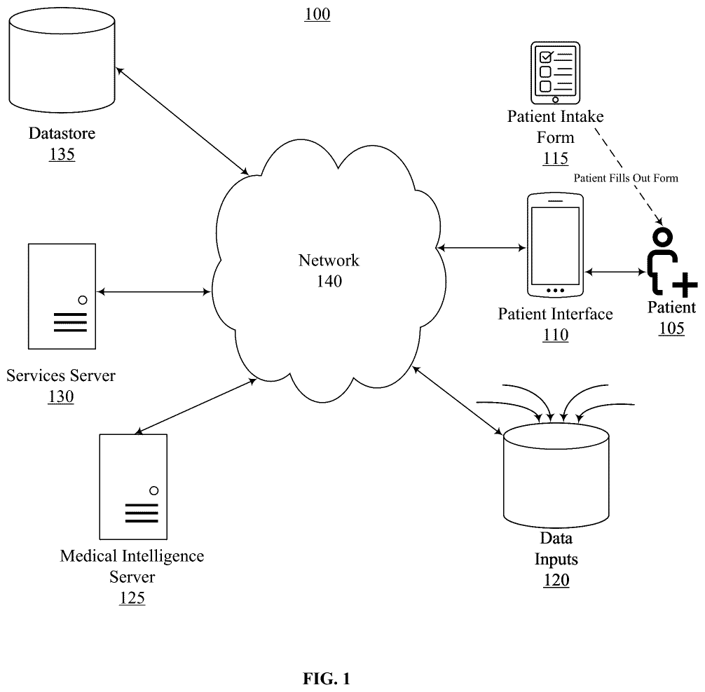

FIG. 1 is a diagram illustrating an exemplary action oriented medical guidance platform 100 in which some embodiments may operate. The guidance platform 100 may comprise a patient 105, a patient interface 110, data inputs 120, medical intelligence server 125, services server 130, datastore 135 and network 140. Patient 105 may be provided with a patient intake form 115 to be filled out. The patient intake form may be completed by the patient on the patient interface 110.

Data inputs 120 may comprise patient information from a plurality of sources. The information may be aggregated before or after retrieval from the plurality of different sources. The collected and/or retrieved data may be stored in datastore 135 and accessed and used by the patient interface 105, medical intelligence server 125 and services server 130.

Medical intelligence server 125 may be one or more physical or virtual machines configured to communicate with the patient interface 110, data inputs 120, services server 130, and datastore 135. The one or more servers may be configured as a distributed computing infrastructure and processing of applications and other software may be carried out on the cloud.

Services server 130 may be one or more physical or virtual machines configured to communicate with the patient interface 110, data inputs 120, medical intelligence server 125, and datastore 135. The one or more servers may be configured as a distributed computing infrastructure and processing of applications and other software may be carried out on the cloud. Medical intelligence server 125 and services server 130 may be the same or different physical or virtual machines.

Datastore 135 may be any storage device capable of storing data for processing or as a result of processing information at the patient interface 110, data inputs 120, medical intelligence server 125, and services server 130. The datastore 135 may be a separate device or the same device as medical intelligence server 125 or services server 130. The datastore 115 may be located in the same location as that of medical intelligence server 125 or services server 130, or at separate locations.

Network 140 may be an intranet, internet, mesh, LTE, GSM, peer-to-peer or other communication network that allows the patient interface 110, data inputs 120, medical intelligence server 125, services server 130 and datastore 135 to communicate with one another.

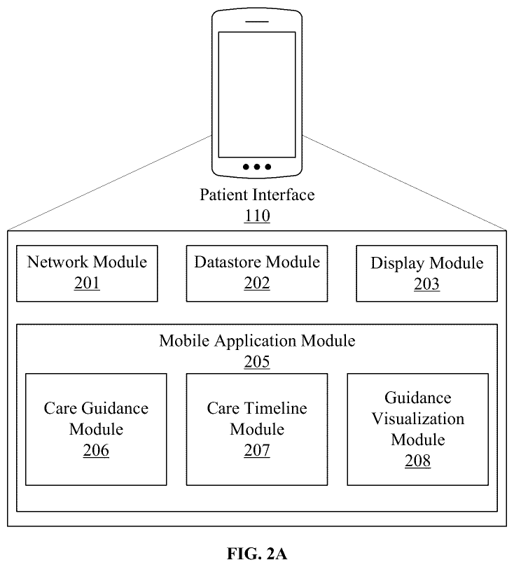

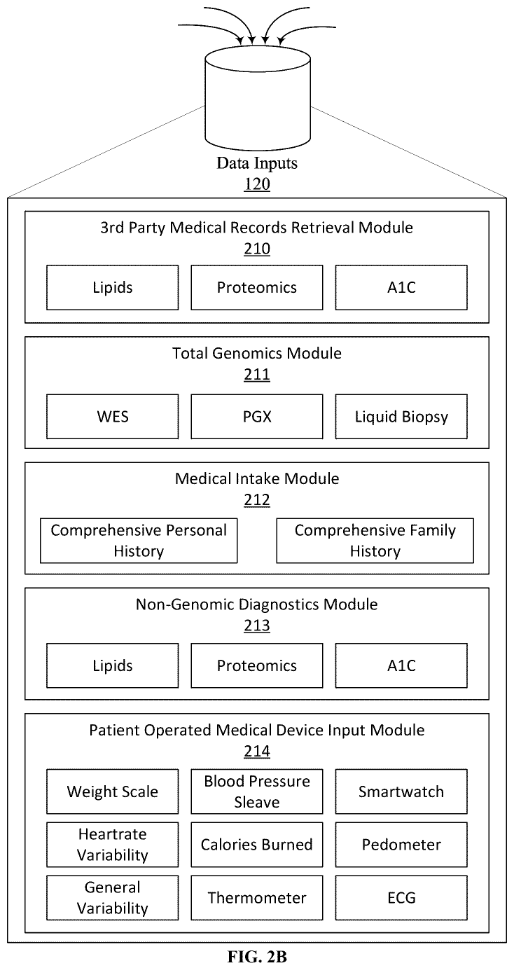

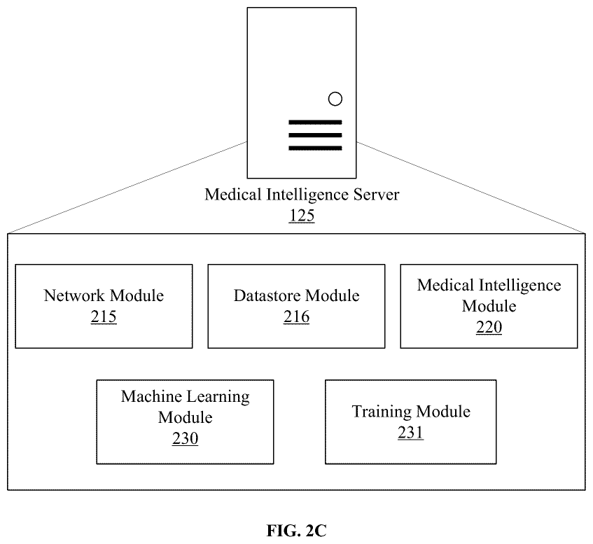

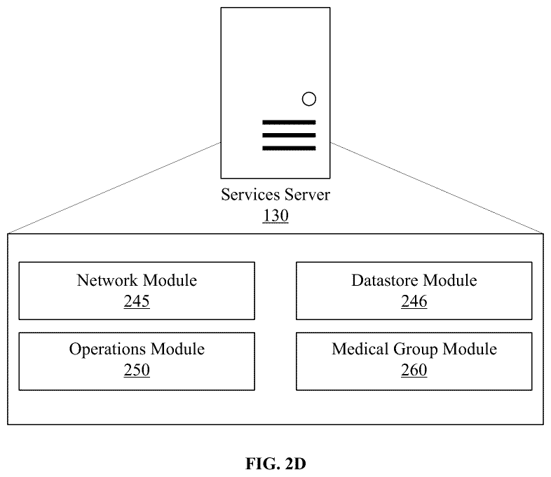

FIG. 2A is a diagram illustrating an exemplary patient interface 110 in accordance with aspects of the present disclosure. Patient interface 110 may comprise network module 201, datastore module 202, display module 203, and mobile application module 205.

Network module 201 may transmit and receive data from other computing systems and devices via a network 140. In some embodiments, the network module 201 may enable transmitting and receiving data from the Internet. Data received by the network module 201 may be used by the other modules of FIG. 2A. The modules may transmit data through the network module 201.

Datastore module 202 may be any hardware or software component capable of storing and retrieving data. In some embodiments, the datastore module may be a hard disk drive, solid state memory, or other memory storage technologies.

Display module 203 may be any device capable of generating a graphical image. In some embodiments, the display module may comprise an LCD, TFT, DLP, OLED or other type of display device. In some embodiments, the display module may comprise a touch screen or digitizer configured to receive input form a patient. Other user input devices may also be used in conjunction with the display interface 110 and/or display module 203.

Mobile application module 205 may comprise care guidance module 206, care timeline module 207 and guidance visualization module 208. Mobile application module 205 may be configured to generate an action item based user interface from which a patient may interact. The user interface may comprise one or more action items. Each action item may be displayed in one or more manners depending on a patient selection, priority, status, risk category or due date. In some embodiments, each action item may be displayed in a compact view and upon selection of an action item by a patient, a detailed view of the action item may be displayed.

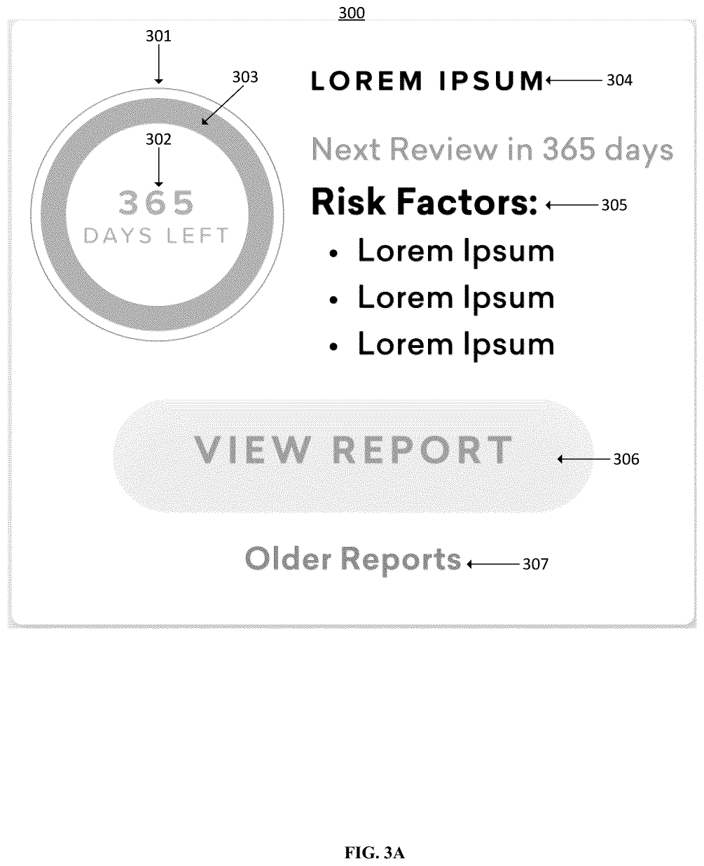

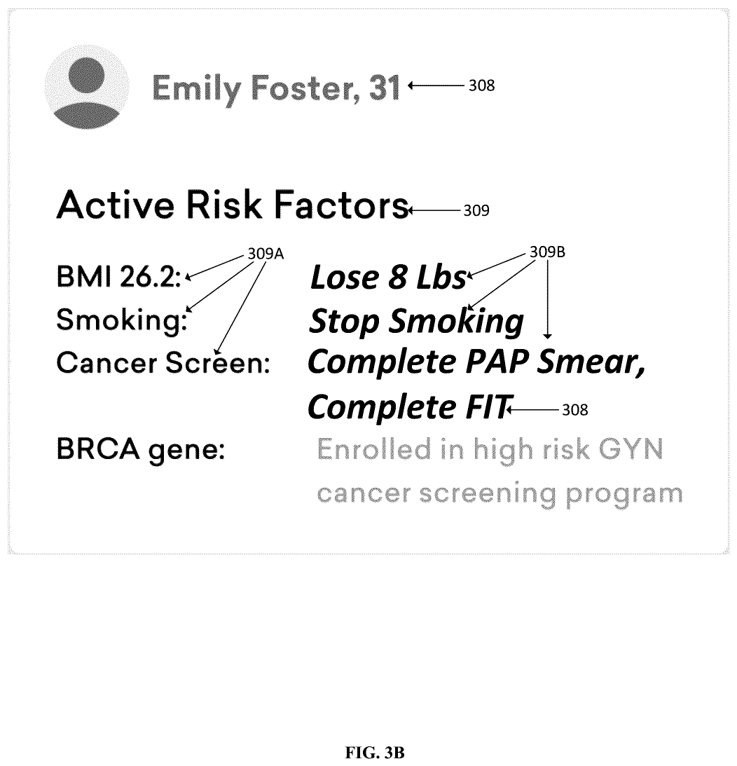

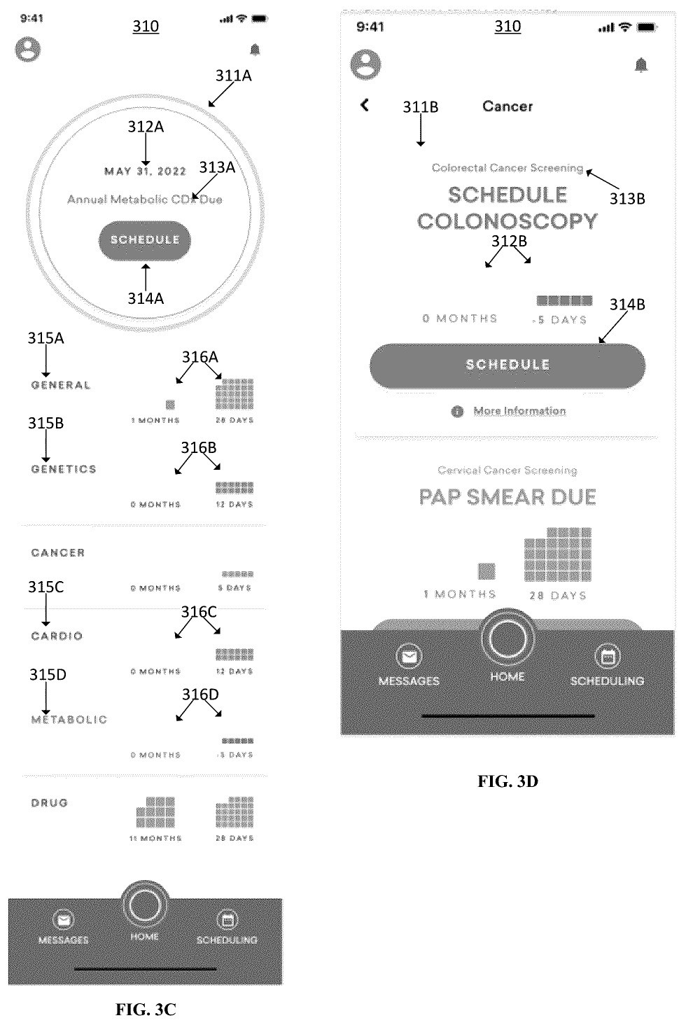

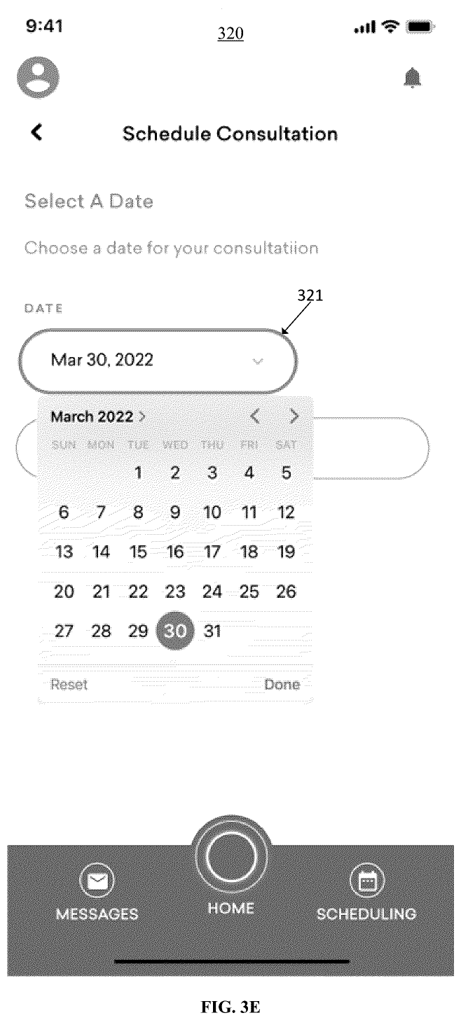

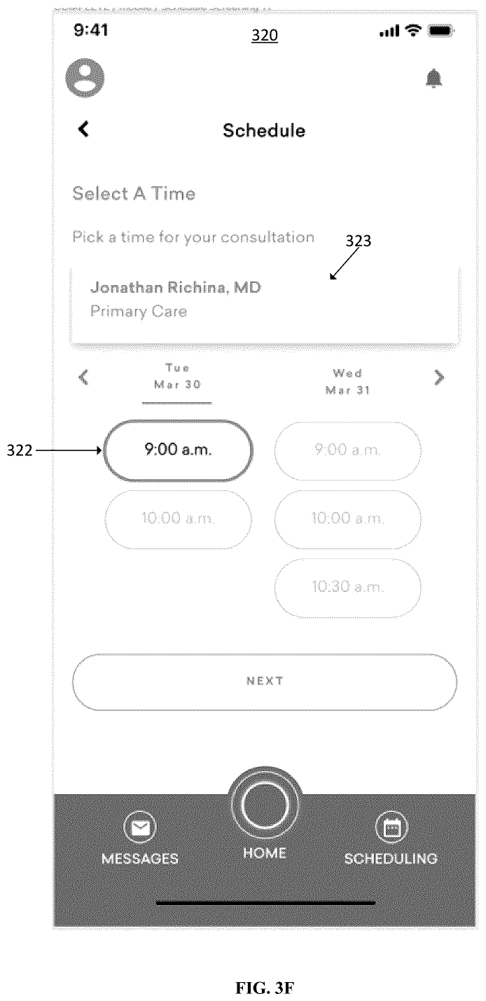

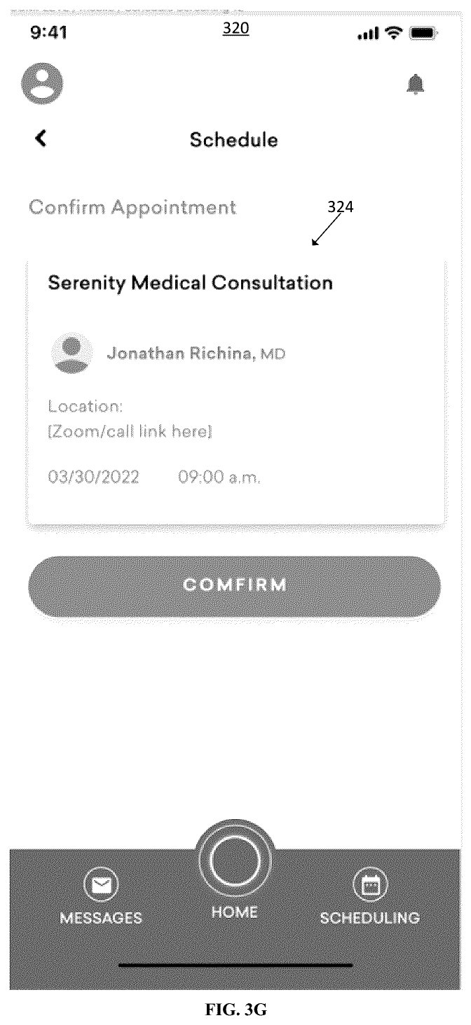

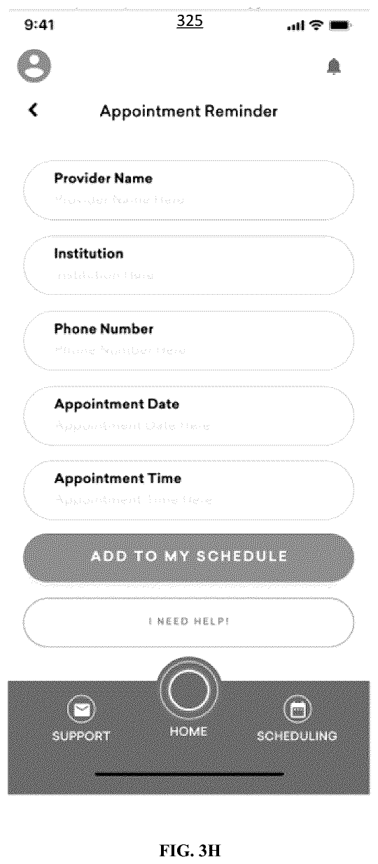

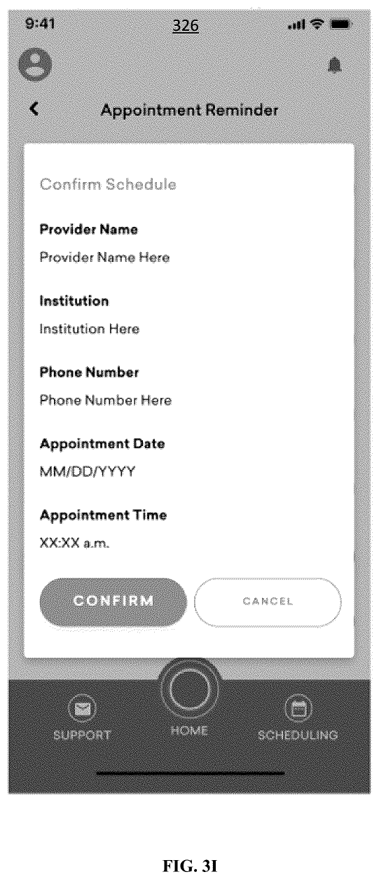

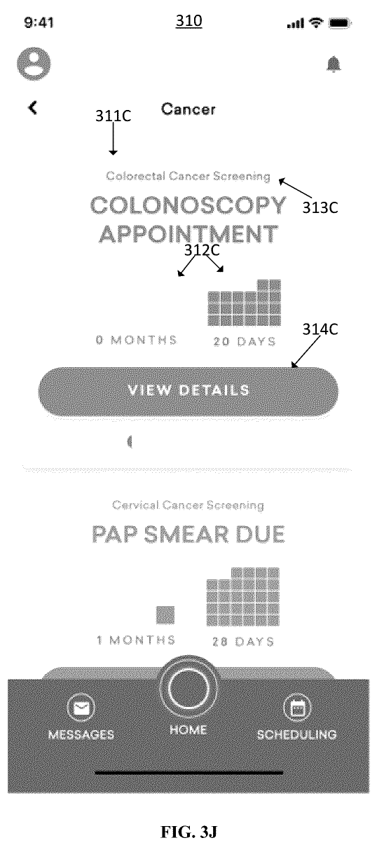

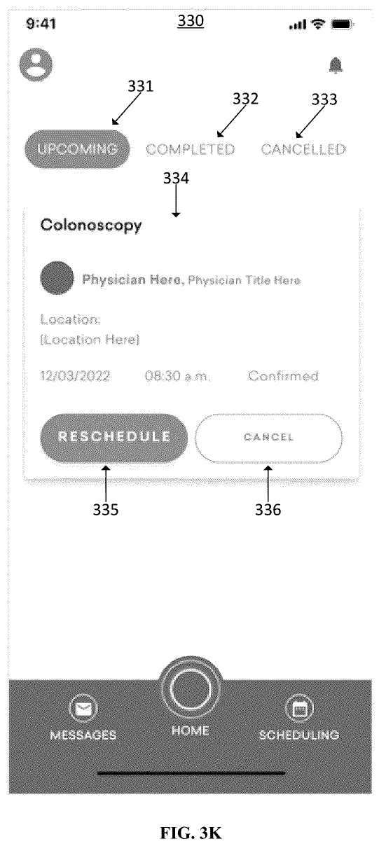

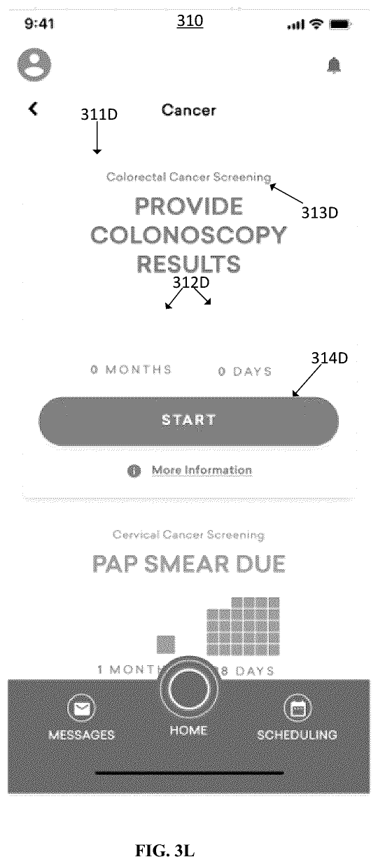

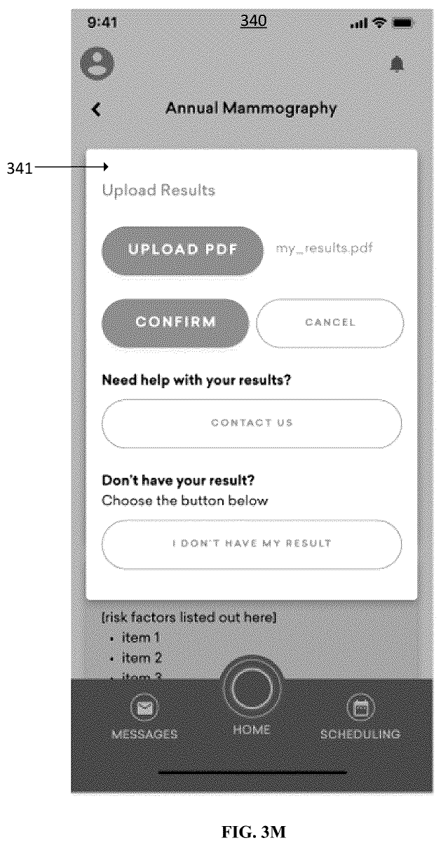

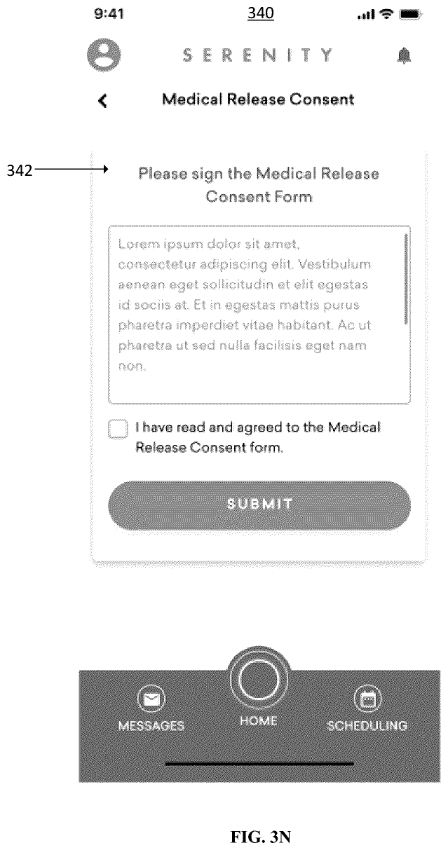

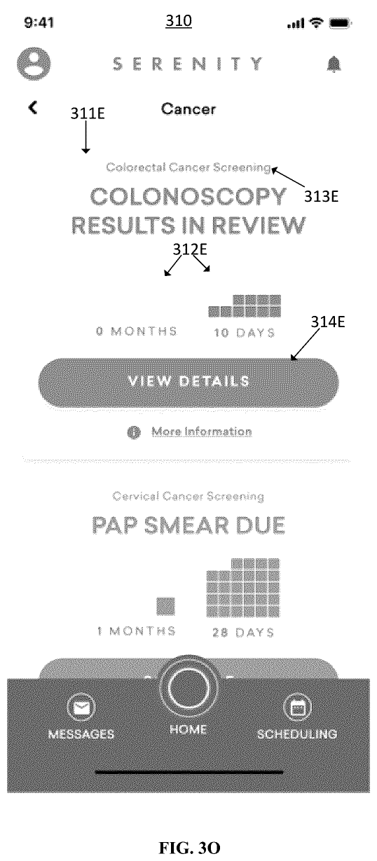

In some embodiments, an action item may be generated for each medical condition or disorder that a patient is diagnosed with. Action items may also be generated for one or more general health categories and/or one or more personalized health categories. For example, there may be action items corresponding to a general health category, a genetics category, a cancer category, a cardio category and a metabolic category as is shown in FIG. 3C. In some embodiments, the action item categories may be implemented as a parent container, wherein the parent container may hold one or more child action items. For example, a cancer category action item may be selected by the patient, and upon selection, one or more child action items may be displayed as is shown in FIG. 3J. In the example, child action items corresponding to colorectal cancer screening and cervical cancer screening are displayed as they belong to and/or are held by the cancer category action item.

Care guidance module 206 may be configured to provide guidance to a patient. The care guidance module 206 may further be configured to encode a patient's personalized preventative care. In some embodiments, the care guidance module 206 may received recommendations and physician decisions from the medical intelligence server 125 or services server 130. Patient specific care guidance may then be generated for an action item based on the received recommendations and physician decisions. The care guidance module 206 may then associate the generated care guidance with the corresponding action item.

Care timeline module 207 may be configured track due dates corresponding to each action item. The care timeline module 207 may determine a due date or timeline for each action item. The due date or timeline may be based on information received from the care guidance module 206. In some embodiments, the care timeline module 207 may also be configured to interface with one or more scheduling system, to allow a patient to schedule, cancel, change or manage appointments corresponding to each action item.

Guidance visualization module 208 may be configured to generate a visual representation of each action item. The visualization may textually or graphically convey information corresponding to the action item, including care guidance, due date and/or countdown timer, risk category, priority and status.

FIG. 2B is a diagram illustrating an exemplary data input 120 in accordance with aspects of the present disclosure. Data input 120 may comprise 3rd party medical records retrieval module 210, total genomics module 211, medical intake module 212, non-genomic diagnostics module 213 and patient operated medical device input module 214.

3rd Party Medical Records Retrieval Module 210 may further comprise lipid test results, proteomics, A1C levels and other test result values. Total Genomics Module 211 may comprise whole exome sequencing (WES) data, Pharmacogenetics (PGx) data and liquid biopsy results data. Medical Intake Module 212 may comprise a comprehensive personal history and a comprehensive family history. Non-Genomic Diagnostics Module 213 may further comprise lipid test results, proteomics, A1C levels and other test result values.

Patient operated medical device input module 214 may be configured to receive patient information from patient operated medical devices. Patient operated medical devices may include weight scales, blood pressure sleeve, smartwatches, fitness trackers, sleep trackers, smart beds, pulse oximeters, pedometers, thermometers, electrocardiograms, electromyograms, electroencephalograms, capnograms, ultrasound sensors, CPAP machines, sleep apnea monitors, blood glucose monitors or any sensor configured to measure patient health. In some embodiments, one or more sensors may be configured to measure heartrate variability, calories burned, steps taken and general variability.

In some embodiments, the one or more patient operated medical devices may be configured to communicate with one or more devices of the patient or directly to the server for analysis. The patient devices may collect data from the patient operated devices and then transfer the data to the server for analysis and review by medical practitioners.

FIG. 2C is a diagram illustrating an exemplary medical intelligence server 125 in accordance with aspects of the present disclosure. Medical intelligence server 125 may comprise network module 215, datastore module 216, medical intelligence module 220, machine learning module 230 and training module 231.

Network module 215 and datastore module 216 may be the same or similar to that of network module 201 and datastore module 202 of FIG. 2A and will not be described for the sake of brevity.

Medical intelligence module 220 may be configured to process genomics data received from data inputs 120. In some embodiments, medical intelligence module may be configured to aggregate and/or unify all or some of the data sources of data inputs 120. In some embodiments, the medical intelligence module 220 may be used to develop one or more personalized risk profiles for a patient. The risk profiles may be based at least in part on the information received from data inputs 120 and the processing of a patient's genomics data. In some embodiments, the risk profiles may be analyzed and matched to one or more evidence-based decision models. In some embodiments, the medical intelligence module 220 may further communicate with patient interface 110 and provide the mobile application module 205 with preventative care and medical guidance decisions, recommendations and analysis models.

Machine learning module 230 may be configured to learn from received physician decisions and inputs corresponding to a proposed risk profile, risk categorization or recommendation generated by one or more machine learning models operating on the machine learning module 230. The one or more machine learning models may be trained by the training module 231 on one or more sets of training data. The training data may be updated with newly generated data sets corresponding to a patient. The new data sets may comprise patient data, predictions, recommendations and classifications generated by the one or more machine learning models, and physician decisions (labeling) corresponding to the predictions, recommendations and classifications.

FIG. 2D is a diagram illustrating an exemplary services server in accordance with aspects of the present disclosure. Services server 130 may comprise network module 245, datastore module 246, operations module 250 and medical group module 260.

Network module 245 and datastore module 246 may be the same or similar to that of network module 201 and datastore module 202 of FIG. 2A and will not be described for the sake of brevity.

Operations module 250 may be configured to coordinate the retrieval of patient records, nurse scheduling, procedure scheduling and provider identification.

Medical group module 260 may be configured for real-time review of a patient's status and results. Medical group module 260 may also be configured to receive patient risk profiles generated by the medical intelligence server 125. The risk profiles may be generated under consideration of all data and medical evidence across tens of millions of data points and millions of preventative care scenarios.

FIG. 3A is a diagram illustrating an exemplary user interface 300 in accordance with some embodiments. User interface 300 may be configured to display an action item or information relating to a patient medical condition or disorder. As shown in FIG. 3A, an action item indicator 301 may be displayed to provide a user with an intuitive representation of data related to an action item. There may be a text due date indicator 302 and/or a graphical countdown timer 303. Text due date indicator 302 may be configured to display a due date or a textual countdown timer. In some embodiments, the textual countdown timer may display the number of days until the due date or the number of days overdue (negative number). In some embodiments, the text due date indicator 302 may be configured to display both the due date itself (including all forms of month/day/year representation) and the textual countdown timer. In some embodiments, any combination of text due date indicator 302 and graphical countdown timer 303 may be displayed at the same time. For example, the action item indicator 301 may be configured to display the due date, a textual countdown timer and a visual representation of the countdown timer.

In some embodiments, graphical countdown timer 303 may be displayed as a scale of colors representing a how much time is left until the due date. For example, the color of the graphical countdown timer 303 may be displayed as a first color (green) when the due date is further away than a first predetermined threshold number of days (i.e. 90 days) and when the due date is within a second predetermined threshold number of days and/or overdue (i.e. 10 days), the graphical countdown timer 303 may be displayed as a second color (red). In some embodiments, there may be one or more intermediate colors and associated thresholds to visually represent a time until the due date. In some embodiments, the graphical countdown timer 303 user other visual representation of numbers, percentages or progress. For example, in FIG. 3A, graphical countdown timer 303 may be a ring shaped progress bar, wherein the fullness and color may change as the number of days decrease. The ring shaped progress bar is shown as a completely full bar in FIG. 3A, corresponding to an initial number of days before the due date. A proportional amount of the ring shaped progress bar may be removed or adjusted to corresponding to the passage of time.

User interface 300 may also comprise a title 304, wherein the title corresponds to a medical condition, disorder, procedures, tests, screening or courses of action. The user interface 300 may also be configured to display one or more risk factors corresponding to the action item and allow the patient to view a detailed report for the displayed action item. View report button 306 and older reports button 307 may allow the patient to request and view both current and historic reports.

FIG. 3B is a diagram illustrating an exemplary user interface in accordance with some embodiments. In some embodiments, FIG. 3B represents the details displayed by the user interface in response to the patient selecting the view report button 306 of FIG. 3A. In some embodiments, a detailed report associated with the action item may be displayed. The displayed details may include the patients name 308 and active risk factors 309. In some embodiments, a list of active risk factors 309A and their corresponding recommended medical interventions 309B may be displayed to the patient. The active risk factors 309A and their corresponding medical interventions 309B may be organized by risk category and/or priority. In some embodiments, the priority and risk category of the active risk factors 309A and their corresponding medical interventions 309B may be color coded to provide a more intuitive understanding of the importance of each active risk factor.

FIG. 3C is a diagram illustrating an exemplary action item based user interface 310 in accordance with some embodiments. Action item based user interface 310 may comprise a detailed action item indicator 311A further comprising a text due date indicator 312A, an action item title 313A a schedule button 314A. The interface may further include one or more preview tiles, 315A-315D, representing action items. The patient may select a preview tile to view an action item indicator for the action item corresponding to the selected preview tile. A patient may select any action item from the interface and schedule a medical intervention corresponding to the selected action item. A schedule button 314A may allow a user to initiate a scheduling procedure for each action item individually.

Preview tile 315A shows an exemplary action item “General” along with a graphical countdown indicator showing the number of days until the due date of the action item. The graphical countdown indicator 316A may be colored green to indicate that the due date is outside of a threshold number of days away (i.e. 30 days).

Preview tile 315B shows an exemplary action item “Genetics” along with a graphical countdown indicator 316B. The graphical countdown indicator 316B may be colored blue to indicate that the action item is in review.

Preview tile 315C shows an exemplary action item “Cardio” along with a graphical countdown indicator 316C. The graphical countdown indicator 316C may be colored orange to indicate the due date is within a threshold number of days away (i.e. 30 days).

Preview tile 315D shows an exemplary action item “Metabolic” along with a graphical countdown indicator 316D. The graphical countdown indicator 316D may be colored red to indicate an overdue due date.

In some embodiments, the color of the graphical countdown indicators 316A-316D may be based on the number of days away from the due date, the risk factor associated with the action item, or status of the action item.

FIG. 3D is a diagram illustrating an exemplary action item based user interface 310 in accordance with some embodiments. The detailed action item indicator 311B may be the same or similar to that of 311A of FIG. 3C, but with a square tile based detail view instead of the round detail view of detailed action item indicator 311A.

FIG. 3E-3G are diagrams illustrating an exemplary action item based scheduling interface 320 in accordance with some embodiments. The action item based scheduling interface may be configured to allow a patient to select, for an action item, a requested date 321, requested time 322 and requested doctor 323 for an appointment. After selection of the requested date 321, requested time 322 and requested doctor 323, an appointment request 324 is created and may then be confirmed and submitted by the patient.

FIGS. 3H-3I are diagrams illustrating an exemplary action item based appointment interface 325 in accordance with some embodiments. As shown in FIG. 3H, the appointment interface 325 may be configured to show details related to a scheduled appointment generated in FIG. 3E. The interface may be configured to add the scheduled appointment to a calendar system or other organizational system to track user appointments. The interface may be configured to confirm the addition of the scheduled appointment before it is added to the calendar system or tracking system.

FIG. 3J is a diagram illustrating an exemplary action item based user interface 310 after performing a scheduling operation for the action item in accordance with some embodiments. The detailed action item indicator 311C shows the same or similar information to that of 311B of FIG. 3D. The difference being that the graphical visualizations of details of the action item are updated to represent an update in the status of the action item. For example, the detailed action item indicator 311B in FIG. 3D may be colored red to signify that the action item is overdue. After scheduling of an appointment for the action item, the detailed action item indicator 311C may be displayed in a blue color, signifying that the action item is pending/in review. The status may be changed from “SCHEDULE COLONOSCOPY” in FIG. 3D to “COLONOSCOPY APPOINTMENT” and the textual countdown indicator and graphical countdown indicator may be updated based on the date of the scheduled appointment. The patient may request to see or change the details of the appointment. In some embodiments, a selection button such as view details button 314C may be used to initiate a review or change to the appointment.

FIG. 3K is a diagram illustrating an exemplary action item based appointment calendar 330 after performing a scheduling operation for the action item in accordance with some embodiments. Appointment calendar 330 may comprise tabs or sections for displaying upcoming appointments 331, completed appointments 332 and canceled appointments 333. Appointment details 334 may be shown for a selected appointment. The selection details may include a function to reschedule 335 or cancel 336 the appointment.

FIG. 3L is a diagram illustrating an exemplary action item based user interface 310 after the patient has completed a scheduled appointment corresponding to the action item. The user interface may visually indicate (colored red) that the action item is waiting for a patient to perform one or more actions. For example, detailed action item indicator 311D is colored red, and the status has been changed to “PROVIDE COLONOSCOPY RESULTS.” The patient may then begin the process of providing the required information to the interface. In some embodiments, the patient may click the start button 314D to initiate the transfer of results. FIG. 3M shows an exemplary result transfer interface 340. An upload selection box 341 may be displayed to the patient and be configured to accept patient data and results in the form of a pdf or other file format.

FIG. 3N is a diagram illustrating an exemplary result transfer interface 340. The result transfer interface 340 may be configured to import the results from the facility that the appointment was held at. In some embodiments, a medical release form 342 may be electronically signed by the patient to allow the results to be transferred or otherwise shared with the user interface, patient interface 110, data sources 120, the medical intelligence server 125, services server 130 and datastore 135.

FIG. 3O is a diagram illustrating an exemplary action item based user interface 310 after the patient has uploaded the results of the appointment. As shown, the status of the action item has been updated to “COLONOSCOPY RESULTS IN REVIEW,” and detailed action item indicator 311E is displayed in a blue color to indicate that the status of the action item is in review or is otherwise waiting for action from a physician or lab.

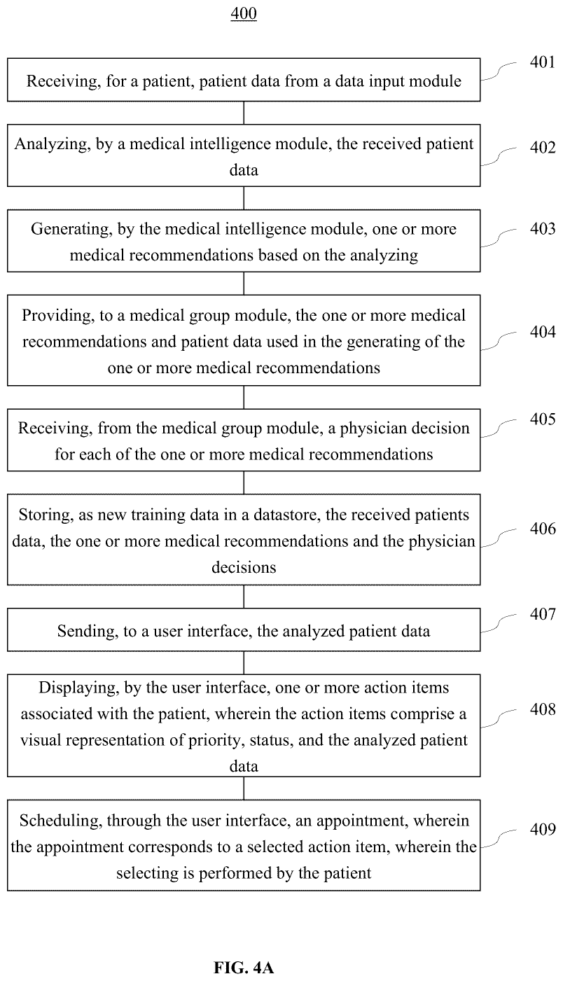

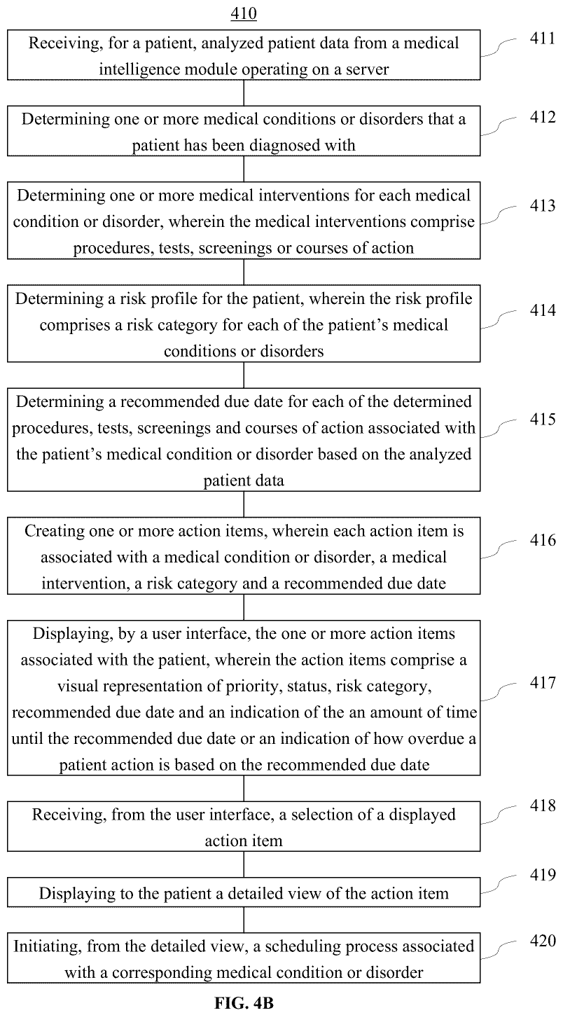

FIG. 4A is a flow chart illustrating an exemplary method 300 that may be performed in accordance with some embodiments.

At step 401, medical guidance system is configured to receive, for a patient, patient data from a data input module.

At step 402, medical guidance system is configured for analyzing, by a medical intelligence module, the received patient data.

At step 403, medical guidance system is configured to generate, by the medical intelligence module, one or more medical recommendations based on the analyzing.

At step 404, medical guidance system is configured to provide, to a medical group module, the one or more medical recommendations and patient data used in the generating of the one or more medical recommendations.

At step 405, medical guidance system is configured to receive, from the medical group module, a physician decision for each of the one or more medical recommendations.

At step 406, medical guidance system is configured to store, as new training data in a datastore, the received patient data, the one or more medical recommendations and the physician decisions.

At step 407, medical guidance system is configured send, to a user interface, the analyzed patient data.

At step 408, medical guidance system is configured to display, by the user interface, one or more action items associated with the patient, wherein the action items comprise a visual representation of priority, status, and the analyzed patient data.

At step 409, medical guidance system is configured to scheduling, through the user interface, an appointment, wherein the appointment corresponds to a selected action item, wherein the selecting is performed by the patient.

FIG. 4B is a flow chart illustrating an exemplary method 410 that may be performed in accordance with some embodiments.

At step 411, the medical guidance user interface is configured to receive, for a patient, analyzed patient data from a medical intelligence module operating on a server;

At step 412, the medical guidance user interface is configured to determine one or more medical conditions or disorders that a patient has been diagnosed with.

At step 413, the medical guidance user interface is configured to determine one or more medical interventions for each medical condition or disorder, wherein the medical interventions comprise procedures, tests, screenings or courses of action.

At step 414, the medical guidance user interface is configured to determine a risk profile for the patient, wherein the risk profile comprises a risk category for each of the patient's medical conditions or disorders.

At step 415, the medical guidance user interface is configured to determine a recommended due date for each of the determined procedures, tests, screenings and courses of action associated with the patient's medical condition or disorder based on the analyzed patient data.

At step 416, the medical guidance user interface is configured to creating one or more action items, wherein each action item is associated with a medical condition or disorder, a medical intervention, a risk category and a recommended due date.

At step 417, the medical guidance user interface is configured to display, by a user interface, the one or more action items associated with the patient, wherein the action items comprise a visual representation of priority, status, risk category, recommended due date and an indication of the an amount of time until the recommended due date or an indication of how overdue a patient action is based on the recommended due date.

At step 418, the medical guidance user interface is configured to receive, from the user interface, a selection of a displayed action item.

At step 419, the medical guidance user interface is configured to display to the patient, through the user interface, a detailed view of the action item.

At step 420, the medical guidance user interface is configured to initiate, from the detailed view, a scheduling process associated with the corresponding action item's medical condition or disorder.

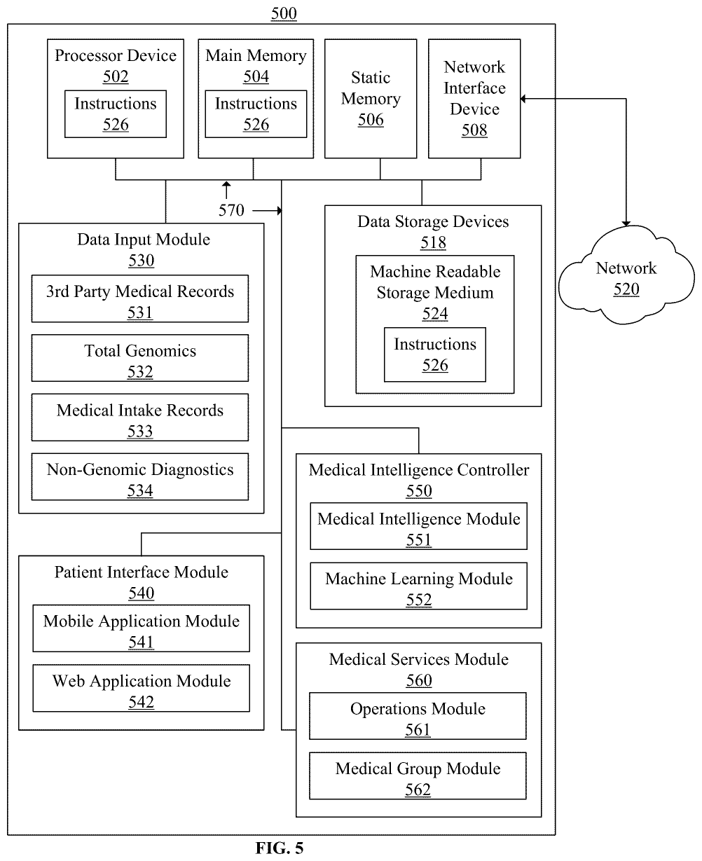

FIG. 5 illustrates an example machine of a computer system within which a set of instructions, for causing the machine to perform any one or more of the methodologies discussed herein, may be executed. In alternative implementations, the machine may be connected (e.g., networked) to other machines in a LAN, an intranet, an extranet, an ad-hoc network, a mesh network, and/or the Internet. The machine may operate in the capacity of a server or a client machine in client-server network environment, as a peer machine in a peer-to-peer (or distributed) network environment, or as a server or a client machine in a cloud computing infrastructure or environment.

The machine may be a personal computer (PC), a tablet PC, a set-top box (STB), a Personal Digital Assistant (PDA), a cellular telephone, a web appliance, a server, a network router, a switch or bridge, or any machine capable of executing a set of instructions (sequential or otherwise) that specify actions to be taken by that machine. Further, while a single machine is illustrated, the term “machine” shall also be taken to include any collection of machines that individually or jointly execute a set (or multiple sets) of instructions to perform any one or more of the methodologies discussed herein.

The example computer system 500 includes a processing device 502, a main memory 504 (e.g., read-only memory (ROM), flash memory, dynamic random access memory (DRAM) such as synchronous DRAM (SDRAM) or Rambus DRAM (RDRAM), etc.), a static memory 506 (e.g., flash memory, static random access memory (SRAM), etc.), and a data storage device 518, which communicate with each other via a bus 560.

Processing device 502 represents one or more general-purpose processing devices such as a microprocessor, a central processing unit, or the like. More particularly, the processing device may be complex instruction set computing (CISC) microprocessor, reduced instruction set computing (RISC) microprocessor, very long instruction word (VLIW) microprocessor, or processor implementing other instruction sets, or processors implementing a combination of instruction sets. Processing device 502 may also be one or more special-purpose processing devices such as an application specific integrated circuit (ASIC), a field programmable gate array (FPGA), a digital signal processor (DSP), network processor, or the like. The processing device 502 is configured to execute instructions 526 for performing the operations and steps discussed herein.

The computer system 500 may further include a network interface device 608 to communicate over the network 520. The computer system 500 also may include sensor array 510. Sensor array 510 may comprise a camera sensor 512, infrared sensor 514 and depth sensor 516.

The data storage device 518 may include a machine-readable storage medium 524 (also known as a computer-readable medium) on which is stored one or more sets of instructions or software 526 embodying any one or more of the methodologies or functions described herein. The instructions 526 may also reside, completely or at least partially, within the main memory 504 and/or within the processing device 502 during execution thereof by the computer system 500, the main memory 504 and the processing device 502 also constituting machine-readable storage media.

Data input module unit 530 may comprise 3rd party medical records 531, total genomics 532, medical intake records 533 and non-genomic diagnostics 534. Data input module 530 may be similar to that of data input 120 of FIGS. 1 and 2B. 3rd party medical records 531, total genomics 532, medical intake records 533 and non-genomic diagnostics 534 may be the same or similar to that of 3rd party medical records retrieval module 210, total genomics module 211, medical intake module 212 and non-genomic diagnostics module 213 of FIG. 2B.

Patient interface module 540 may comprise mobile application module 541 and web application module 542. Mobile application module 541 may be the same or similar to that of mobile application module 205 of FIG. 2A. In some embodiments, web application module may be the similar to that of mobile application modules 541 and 205 and will not be described for the sake of brevity.

Medical intelligence controller 550 may comprise medical intelligence module 551 and machine learning module 552. Medical intelligence module 551 and machine learning module 552 may be the same or similar to that of medical intelligence module 220 and machine learning module 230 of FIG. 2C and will not be described for the sake of brevity.

Medical services module 560 may comprise operations module 561 and medical group module 562. Operations module 561 and medical group module 562 may be the same or similar to that of operations module 250 and medical group module 260 of FIG. 2D and will not be described for the sake of brevity.

In one implementation, the instructions 526 include instructions to implement functionality corresponding to the components of a device to perform the disclosure herein. While the machine-readable storage medium 524 is shown in an example implementation to be a single medium, the term “machine-readable storage medium” should be taken to include a single medium or multiple media (e.g., a centralized or distributed database, and/or associated caches and servers) that store the one or more sets of instructions. The term “machine-readable storage medium” shall also be taken to include any medium that is capable of storing or encoding a set of instructions for execution by the machine and that cause the machine to perform any one or more of the methodologies of the present disclosure. The term “machine-readable storage medium” shall accordingly be taken to include, but not be limited to, solid-state memories, optical media and magnetic media.

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as “identifying” or “determining” or “executing” or “performing” or “collecting” or “creating” or “sending” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage devices.

The present disclosure also relates to an apparatus for performing the operations herein. This apparatus may be specially constructed for the intended purposes, or it may comprise a general purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions, each coupled to a computer system bus.

Various general purpose systems may be used with programs in accordance with the teachings herein, or it may prove convenient to construct a more specialized apparatus to perform the method. The structure for a variety of these systems will appear as set forth in the description above. In addition, the present disclosure is not described with reference to any particular programming language. It will be appreciated that a variety of programming languages may be used to implement the teachings of the disclosure as described herein.

The present disclosure may be provided as a computer program product, or software, that may include a machine-readable medium having stored thereon instructions, which may be used to program a computer system (or other electronic devices) to perform a process according to the present disclosure. A machine-readable medium includes any mechanism for storing information in a form readable by a machine (e.g., a computer). For example, a machine-readable (e.g., computer-readable) medium includes a machine (e.g., a computer) readable storage medium such as a read only memory (“ROM”), random access memory (“RAM”), magnetic disk storage media, optical storage media, flash memory devices, etc.

In the foregoing disclosure, implementations of the disclosure have been described with reference to specific example implementations thereof. It will be evident that various modifications may be made thereto without departing from the broader spirit and scope of implementations of the disclosure as set forth in the following claims. The disclosure and drawings are, accordingly, to be regarded in an illustrative sense rather than a restrictive sense.

What is claimed is:

1. A computer implemented medical action guidance system method comprising:
receiving, at a server, patient data from a data input module for a patient, wherein the patient data comprises:
comprehensive personal history;
comprehensive family history;
third party medical records;
total genomics records;
non-genomics diagnostic records;
analyzing, by a medical intelligence module operating on the server, the received patient data, wherein the analyzing is performed by a machine learning model, and wherein the machine learning model is trained, by a training module, on labeled datasets comprising historic patient data, medical recommendations and physician decisions;
generating, by the medical intelligence module, one or more medical recommendations based on the analyzing;
providing, to a medical group module operating on the server, the one or more medical recommendations and patient data used in the generating of the one or more medical recommendations;
receiving, from the medical group module, a physician decision for each of the one or more medical recommendations;
storing, as new training data in a datastore, the received patients data, the one or more medical recommendations and the physician decisions;
sending, to a patient interface operating on a patient device, the analyzed patient data;
displaying, by the user interface, one or more action items associated with the patient, wherein the action items comprise a visual representation of priority, status, and the analyzed patient data;
ordering the action items based on priority and status; and
scheduling, through the user interface, an appointment based at least in part on patient input, wherein the appointment corresponds to a selected action item, wherein the selecting is performed by the patient.

2. The method of claim 1, wherein the comprehensive personal history and the comprehensive family history are obtained from the patient through a patient intake form.

3. The method of claim 1, wherein the analyzing by the medical intelligence module further comprises:
classifying, by a risk profile module, a patient risk profile, wherein the risk profile corresponds to one of a plurality of risk categories; and
wherein the providing of the one or more medical recommendations to the medical group module further comprises providing the classified patient risk profile.

4. The method of claim 1, wherein the visual representation of status corresponds to a completion state of a procedure and a number of days until a due date of the action item.

5. The method of claim 1, wherein the machine learning model is retrained, by the training module, on the new training data.

6. The method of claim 1, wherein the receiving of patient data from the data input module further comprises:
establishing a connection to one or more patient operated medical device input modules, wherein the one or more patient operated medical device input modules include:
a weight scale;
a blood pressure monitor, wherein the blood pressure monitor includes a sleeve sensor unit;
a thermometer;
an electrocardiogram; and
a smartwatch, worn by the patient, wherein the smartwatch includes one or more biosensors and wherein the smartwatch is configured to determine one or more physiological parameters of the patient, the physiological parameters including:
heart rate variability;
general variability;
pulse rate;
pulse oximetry; and
skin conductance response.

7. The method of claim 6, wherein the smartwatch further includes one or more inertial measurement units, and wherein the smartwatch is further configured to:
determine a number of steps taken by the patient based on the one or more inertial measurements units; and
estimating a number of calories burned by the patient based on the determined number of steps taken by the patient and the determined one or more physiological parameters of the patient.

8. A computer implemented interface for medical action guidance configured to perform a method comprising:
receiving, for a patient, analyzed patient data from a medical intelligence module operating on a server;
determining one or more medical conditions or disorders that a patient has been diagnosed with;
determining one or more medical interventions for each medical condition or disorder, wherein the medical interventions comprise procedures, tests, screenings or courses of action;
determining a risk profile for the patient, wherein the risk profile comprises a risk category for each of the patient's medical conditions or disorders
determining a recommended due date for each of the determined procedures, tests, screenings and courses of action associated with the patient's medical condition or disorder based on the analyzed patient data;
creating one or more action items, wherein each action item is associated with a medical condition or disorder, a medical intervention, a risk category and a recommended due date;
displaying, by a user interface on a patient device, the one or more action items associated with the patient, wherein the action items comprise a visual representation of priority, status, risk category, recommended due date and an indication of the an amount of time until the recommended due date or an indication of how overdue a patient action is based on the recommended due date;
receiving, from the user interface, a selection of a displayed action item;
displaying to the patient, through the user interface, a detailed view of the action item;
initiating, from the detailed view, a scheduling process associated with the corresponding action item's medical condition or disorder.

9. The computer implemented interface of claim 8, wherein the patient device is configured to:
establish a connection to one or more patient operated medical devices;
receive, from the one or more patient operated medical devices, one or more physiological parameters for the patient;
create one or more action items corresponding to the received physiological parameters; and
display to the patient, through the user interface, a visual representation of the received physiological parameters.

10. The computer implemented interface of claim 9, wherein the one or more patient operated medical devices includes:
a weight scale;
a blood pressure monitor, wherein the blood pressure monitor includes a sleeve sensor unit;
a thermometer;
an electrocardiogram; and
a smartwatch, worn by the patient, wherein the smartwatch includes one or more biosensors and wherein the smartwatch is configured to determine one or more physiological parameters of the patient, the physiological parameters including:
heart rate variability;
general variability;
pulse rate;
pulse oximetry; and
skin conductance response.

11. A system comprising one or more processors, and a non-transitory computer-readable medium including one or more sequences of instructions that, when executed by the one or more processors, cause the system to perform operations comprising:
receiving, for a patient, analyzed patient data from a medical intelligence module operating on a server;
determining one or more medical conditions or disorders that a patient has been diagnosed with;
determining one or more medical interventions for each medical condition or disorder, wherein the medical interventions comprise procedures, tests, screenings or courses of action;
determining a risk profile for the patient, wherein the risk profile comprises a risk category for each of the patient's medical conditions or disorders
determining a recommended due date for each of the determined procedures, tests, screenings and courses of action associated with the patient's medical condition or disorder based on the analyzed patient data;
creating one or more action items, wherein each action item is associated with a medical condition or disorder, a medical intervention, a risk category and a recommended due date;
displaying, by a user interface on a patient device, the one or more action items associated with the patient, wherein the action items comprise a visual representation of priority, status, risk category, recommended due date and an indication of the an amount of time until the recommended due date or an indication of how overdue a patient action is based on the recommended due date;
receiving, from the user interface, a selection of a displayed action item;
displaying to the patient, through the user interface, a detailed view of the action item;
initiating, from the detailed view, a scheduling process associated with the corresponding action item's medical condition or disorder.

12. The system of claim 11, wherein the patient device is configured to:
establish a connection to one or more patient operated medical devices;
receive, from the one or more patient operated medical devices, one or more physiological parameters for the patient;
create one or more action items corresponding to the received physiological parameters; and
display to the patient, through the user interface, a visual representation of the received physiological parameters.

13. The system of claim 12, wherein the one or more patient operated medical devices includes:
a weight scale;
a blood pressure monitor, wherein the blood pressure monitor includes a sleeve sensor unit;
a thermometer;
an electrocardiogram; and
a smartwatch, worn by the patient, wherein the smartwatch includes one or more biosensors and wherein the smartwatch is configured to determine one or more physiological parameters of the patient, the physiological parameters including:
heart rate variability;
general variability;
pulse rate;
pulse oximetry; and
skin conductance response.
